(19) United States
(12) Patent Application Publication
(10) Pub. No.: US 2022/0400946 A1
(43) Pub. Date: Dec. 22, 2022

(54) VIVO CALIBRATION OF DOPPLER FLOWMETRY

(71) Applicant: Physical Sciences, Inc., Andover, MA (US)

(72) Inventors: Mircea Mujat, Acton, MA (US); Nicusor Iftimia, Windham, NH (US)

(21) Appl. No.: 17/721,728

(22) Filed: Apr. 15, 2022

Related U.S. Application Data
(60) Provisional application No. 63/211,783, filed on Jun. 17, 2021.

(51) Int. Cl. A61B 3/10; A61B 3/12

(52) U.S. Cl. CPC A61B 3/102; A61B 3/1233; A61B 3/1025

(57) ABSTRACT

A method for determining a calibration factor in Doppler flowmetry velocity measurements in the living eye includes imaging the eye with Doppler flowmetry and processing data to obtain blood velocity, volume, and flow maps using Doppler flowmetry formulas that provide velocity as a mean frequency expressed in Hz, and volume and flow in arbitrary units. A selected blood vessel is probed with Doppler OCT to measure the absolute velocity of blood at that location expressed in mm/s to determine a calibration factor used to convert the velocity measured with Doppler flowmetry expressed in Hz to velocity expressed in mm/s.

### RELATED APPLICATIONS

This application claims benefit of and priority to U.S. Provisional Application Ser. No. 63/211,783 filed Jun. 17, 2021, under 35 U.S.C. §§ 119, 120, 363, 365, and 37 C.F.R. § 1.55 and § 1.78, which is incorporated herein by this reference.

### GOVERNMENT RIGHTS

This invention was made with U.S. Government support under Contract No. NNX16CC20C awarded by NASA. The Government may have certain rights in the subject invention.

### FIELD OF THE INVENTION

This invention relates to methods of imaging, for example retinal imaging in order to detect eye diseases.

### BACKGROUND OF THE INVENTION

The leading causes of blindness worldwide (age related macular degeneration (AMD), diabetic retinopathy (DR), and glaucoma) have vascular components that have been investigated for many years. The total number of cases for these three eye diseases in the U.S. (age 50 and older) grew from 8.03 million in 2000 to 12.48 million in 2010 and is expected to rise above 25 million by 2050, with a devastating impact on the quality of life and a staggering economic burden on society. Examination of the retinal vasculature plays an important role in diagnosing eye diseases. However, a critical barrier in understanding the in vivo vascular involvement in these diseases is the lack of non-invasive methods for precise quantification of the vascular flow within the ocular structures.

The retina is highly vascularized and very metabolically active and like the central nervous system of which it is a part, it is susceptible to ischemic (insufficient blood flow) injury. The retina represents the only part of the central nervous system where capillary blood flow is visible and can be measured by non-invasive means. Degenerative neurovascular diseases (e.g., diabetic retinopathy) of the eye often have either hemodynamic consequences or causes, though the mechanisms are poorly understood.

Improved blood flow imaging diagnostics will aid the detection and management of many eye conditions including AMD, DR, and glaucoma. Both the retinal and choroidal vessel diameters range from ˜5 μm (in the retinal capillary bed and choriocapillaris) to ˜0.4 mm (major vessels). Flow velocities range from local quasi-isotropic perfusion of tens of μm/s in the capillaries to pulsatile values of several cm/s in the arteries. This range of dimensions and flow parameters presents an extremely demanding diagnostic problem in terms of spatial resolution, field of view, imaging depth, and dynamic range and currently there is no known instrument on the market that can fulfill these demands.

The retinal circulation originates from the central retinal artery that passes through the optic nerve head (ONH) before branching into superior, inferior, nasal, and temporal arteries, into many smaller vessels, and ultimately, into capillary networks. The underlying choroidal vessels and choriocapillaris beneath the retinal pigment epithelium (RPE) account for approximately 90% of the blood flow nourishing the retina. While rapid flow in the retinal vascular tree is readily visualized, the perfusion of the retina through the micro-vasculature on both sides of the RPE can be important. For eye diseases such as DR and macular degeneration, these structures will exhibit early flow defects or the growth of new vessels triggered by metabolic distress and other factors. There is increasing evidence that ocular blood flow abnormalities are involved in the pathogenesis of glaucoma and that altered ONH blood flow may play a role in the development and progression of glaucoma.

In addition to the known diseases of the eye, there are other causes that can disturb the hemodynamic activity of the retina. Little is known about the ocular and cerebral blood flow during exposure to increasingly hypoxic conditions (insufficient oxygen supply) or, for that matter hypercapnia (too much CO₂). Blood flow alterations in both the brain and the eye occur under the influence of prolonged hypoxia. There is a close correlation between the regulation of blood supply to the brain and to the retina, due to similar vascular regulatory processes. The auto-regulation of blood flow in the eye is clearly exquisitely sensitive to many neurovascular and metabolic signaling systems. Though glucose, oxygen, and CO₂ (fuel, oxidizer, waste) are the most commonly studied, there is new evidence that intraocular/intracranial pressure changes produce responses with potential long-term ocular health consequences.

OCT is a high-resolution cross-sectional imaging modality that is an optical analog of ultrasound imaging. Through Fourier-domain measurement of time delayed back-reflected light, an axial (depth) reflectivity profile of a sample is constructed (A-scan or A-line). Image depth resolution of a few microns (μm) can be achieved using this technique and measurements are performed in vivo and in real time. An incident light beam directed into an eye and scanned in a transverse direction creates a cross-sectional image (B-scan). Multiple adjacent B-scans in a raster pattern can be acquired at high speed to build a 3D data set, which accurately represents the complete reflectivity structure of the sample or tissue. Numerous biomedical applications of OCT have emerged and are continuously evolving. Visualization of cells, microorganisms, brain, and the interior of arteries have been reported with depth and transversal resolution of 1-15 μm.

Because OCT is a coherent interferometric technique, the relative phase of the reflected sample wave at a given range is well known with respect to the reference wave. Moving particles (like blood cells in a blood vessel) will produce a changing phase between successive A-lines which enables quantitative Doppler flow measurements. The sensitivity and dynamic range of such flow measurements can be scaled for the targeted vessel by clever optical design and advanced signal processing techniques, but it is not currently possible to measure flow simultaneously across large areas and the broad range of velocities, vessel orientations, and calibers encountered in the human eye. New techniques have been developed for ocular 3D flow visualization with OCT (phase- and intensity-variance, OCTA) but these are not quantitative.

The confocal line-scanning ophthalmoscope (LSO) (and its semi-quantitative flow visualization variant line-scanning Doppler flowmetry (LSDF)) have been previously described. The LSO uses line illumination and a linear detector for efficient confocal retinal imaging. LSDF involves acquisition of multiple recordings of a stationary line of deeply penetrating coherent light on the retina. Temporal fluctuations arising from the motion (flow) of particles (erythrocytes) are detected with the confocal line detector at high line rates (20-200 kHz) depending on the maximum desired flow speed, while the dwell time on each line sets the minimum flow speed. As this line beam is slowly swept across the fundus, the motion of blood itself acts as the contrast agent, allowing the visualization of the vessels without dye. The fluctuations are converted to frequencies via Fourier transformation to form a representation of the Doppler power spectrum, which in turn is related to the flow speed in the vessels.

Given the parallel nature of line scanning, LSDF is a significantly improved version of the commercially available technology based on point scanning laser Doppler flowmetry. With this novel imaging approach, the retinal vasculature is visualized in a dye-free angiography mode. The accessible Doppler frequency range for blood flow speeds has been significantly expanded. The LSDF technique introduces a new, fast, and efficient approach to Doppler image capture. Yet these are 2D scalar maps with more or less isotropic velocity sensitivity. As an example, FIG. 1 illustrates an ultra-widefield montage of the retinal and choroidal vasculature shown as a Velocity map and covering almost the entire posterior hemisphere of the left eye of a normal volunteer. The image size in FIG. 1 is 130°×115°. The squid-like structures seen at the top and bottom of FIG. 1 are vortex veins. The back of the “head” (opposite to the “tentacles”) blurs out in these images as the vortex vein goes deeper into the sclera. Three of the four vortex veins in FIG. 1 (one on top-left and the two at the bottom) seem to consist of two “squids”. They are actually branches of the same vortex vein that connect deeper into the sclera. Note that because the LSDF has a relatively long depth of focus of approximately 1 mm, the retinal and choroidal vessels both appear in the flowmetry images although the choroidal vasculature is often distinguished by larger vessels with faster moving blood flow. Image augmentation by 3D OCT variance-type visualization and local, direct OCT Doppler flow measurements can help segment these global LSDF flow images as well as provide a true velocity scale (in the range of 1-100 mm/s), time-resolved pulsatile profiles, and resolve directional ambiguity.

Other existing technologies, such as dye angiography and laser Doppler velocimetry (LDV) also have a series of limitations. Although dye angiography is a powerful tool for global visualization of retinal vessel topology, occlusions and, uniquely, leakage, it provides no information on flow velocity. Fluorescein, generally used in ophthalmology to visualize retinal vasculature, and indocyanine green (ICG) used to visualize the choroid, are considered safe drugs; however, there are health risks associated with injecting contrast agents and both dyes can have adverse reactions, including nausea, vomiting, skin rash, or even death although very rare.

Doppler methods have also been proposed for retinal blood flow diagnostics. Initially, a laser beam focused on the retina was used to measure blood flow using laser Doppler flowmetry. Later, CCD fundus imaging (laser speckle imaging or flowgraphy) and flying-spot confocal scanning laser Doppler flowmetry (e.g. Heidelberg Retinal Flowmeter, HRF) were introduced as imaging modalities. At about the same time, time-domain Doppler optical coherence tomography (DOCT), also called optical Doppler tomography (ODT), was introduced to quantify the axial component of the blood flow. Soon after that, the remarkable speed increase provided by spectral-domain OCT lead to the introduction of spectral-domain ODT (SDODT) for blood flow measurement which mostly replaced the slow time-domain version. Several useful strategies such as dual-beam DOCT and narrow bandwidth phase-reference OCT were reported to improve the sensitivity of DOCT. All of these methods are phase-sensitive, and therefore a phase-stable system is necessary for obtaining high-contrast images.

DOCT is also highly dependent on the scanning beam angle, as flow cannot be detected in vessels perpendicular to the incident light beam, and small variation in the incident angle has a profound impact on the measured flow. Furthermore, this method is also very sensitive to motion artifacts. Two recently developed high-contrast in vivo 2-D/3-D microcirculation imaging techniques, with various implementations by multiple research groups, are speckle-variance (SV) and phase-variance (PV) OCT. SV-OCT images microvasculature by calculating the interline or interframe speckle variance of the intensity-based structural OCT images. The PV-OCT method identifies the phase difference between consecutive B-scans of the same transverse position and allows for mapping the microvasculature. A more recent implementation combining phase and intensity variance, OCT angiography (OCTA) was introduced into clinical practice. However, the information provided by these maps is binary, moving vs. non-moving locations, with no information on flow velocity or dynamics within vessels. Slow flow below the “moving” threshold and very rapid flow above the maximum threshold is marked as no flow, while any flow within the threshold range is marked as a vessel. Considering that metabolic demand of the tissue impacts vessel size and flow, precise quantification of flow is paramount to gauging in vivo tissue functionality, determining disease mechanism, progression, and response to treatment. Computational complexity, slow frame-rate, blood vessel shadowing, phase stability, and bulk tissue motion have been considered drawbacks for these techniques.

As described, the improvement in retinal flow visualization has been considerable, however, there is currently no imaging technique that can provide rapid ultra-wide area imaging of retinal and choroidal vasculature non-invasively (without dyes). In spite of decades of research and introduction of several commercially available instruments, retinal blood flow Doppler imaging has not achieved widespread clinical adoption due to critical issues in interpretability, reproducibility, and imaging speed.

### SUMMARY OF THE INVENTION

Despite decades of research and the introduction of several advanced systems for measurement of blood flow, retinal blood flow Doppler imaging diagnostics have not yet achieved wide-spread adoption.

For example, in laser Doppler flowmetry (LDF), the results are given in arbitrary units (Volume and Flow) or Hz (Velocity) since the conversion factor to the correct parameters claimed to be measured were rather elusive so far. We demonstrate here a method to enable quantitative measurement of the blood velocity by determining the calibration factor, the conversion from Hz to mm/s directly in the living eye and for each measurement session, in this manner eliminating the uncertainties associated with standard LDF.

In one preferred example, the problem of Doppler velocimetry calibration is solved by using Doppler OCT simultaneously at the same location in the living eye every time Doppler velocimetry is performed and using the absolute velocity measured with OCT to calibrate the Doppler velocimetry measurement.

The calibration has been demonstrated and extensively tested on microfluidics in the lab and has been implemented and demonstrated on the living eye through testing on volunteers and on rats with macular degeneration.

Preferably, two imaging modalities: line-scanning Doppler flowmetry (LSDF) and optical coherence tomography (OCT) are combined in the same instrument to provide complementary aspects of ocular blood flow. The wide-field LSDF technique enables visualization and mapping of retinal and choroidal blood vessels without additional contrast agents and over large areas. Doppler OCT quantifies precise local flow parameters and provides the proper calibration factors for LSDF. The combination of these two imaging modalities within the same imaging platform enables comprehensive assessment of blood flow in retina and choroid. By using LSDF as a global semi-quantitative (relative) flow visualization technique and OCT as a local quantitative probe, this combination has the potential to efficiently characterize regional net flow.

In one example, parallel scanning in LSDF enables orders of magnitude speed increase as compared to LDF which in turn affords imaging much larger areas and visualization of much faster flow. See U.S. Pat. No. 7,404,640 incorporated herein by this reference. Retinal and choroidal flow is mapped non-invasively without the use of fluorescein or indocyanine green. Simultaneously, DOCT at specific locations provides quantitative flow values. The calibration factor from frequency to absolute velocity is obtained locally with DOCT every time the LSDF measurement is performed, therefore removing subjective factors related to the operator and the subject. The technique has been demonstrated on microfluidic devices covering a velocity range of 1-100 mm/s precisely controlled with a syringe pump and it has been applied to imaging human and rat retina/choroid.

Featured is a method for determining a calibration factor in Doppler flowmetry velocity measurements in the living eye. The exemplary method includes imaging the eye with Doppler flowmetry and processing the data to obtain blood velocity, volume, and flow maps using Doppler flowmetry formulas that provide velocity as a mean frequency expressed in Hz, and volume and flow in arbitrary units. A selected blood vessel is probed with Doppler OCT to measure the absolute velocity of blood at that location expressed in mm/s and a calibration factor is determined in order to convert the velocity measured with Doppler flowmetry expressed in Hz to velocity expressed in mm/s.

The living eye is preferably a human eye or an animal eye. One Doppler flowmetry method is line-scanning Doppler flowmetry (LSDF). The calibration factor can be calculated using LSDF and OCT measurements at the same location in the retina and at the same time.

Probing a selected blood vessel with Doppler OCT preferably includes scanning the OCT beam in a circular pattern, intersecting the blood vessel twice, identified by two spots in the OCT image, measuring the height difference of the two spots, calculating the angle between the blood vessel and the OCT beam using the measured height difference, and calculating the absolute blood velocity using the angle between the blood vessel and the OCT beam and the axial velocity component obtained from Doppler OCT. The circular OCT scan can be generated by scanning an OCT beam on the surface of a cone with the apex in the center of the eye pupil. In one example, the cone angle is approximately 2 degrees. Obtaining the blood volume can include fitting a profile of the Doppler flowmetry data in a plane perpendicular to the blood vessels with a parabolic function, and calculating the diameter of the blood vessel as the distance between the two points where the parabolic fit function is zero. The Doppler flowmetry data is preferably a velocity map. The Doppler flowmetry data is preferably a volume map or a flow map. Calculating the cross-sectional area of the blood vessel can include using a calculated blood vessel diameter. Obtaining the volumetric flow map expressed in mm³/s may comprise multiplying the calibrated velocity map by the calculated cross-sectional area map.

Also featured is a method of determining blood velocity measurements in the living eye, comprising imaging the eye with Doppler flowmetry; obtaining blood velocity, volume, and flow maps using Doppler flowmetry formulas that provide velocity as a mean frequency expressed in Hz, and volume and flow in arbitrary units; probing a selected blood vessel with Doppler OCT to measure the absolute velocity of blood at that location expressed in distance over time; determining a calibration factor to convert the blood velocity measured with Doppler flowmetry expressed in Hz to blood velocity expressed in distance over time; and calculating the blood velocity in distance over time using the calibration factor.

Also featured is a method for determining blood velocity measurements in the living eye comprising imaging the eye with Doppler flowmetry; obtaining velocity, volume, and flow maps using Doppler flowmetry formulas that provide blood velocity as a mean frequency expressed in Hz and volume and flow in arbitrary units; probing a selected blood vessel with Doppler OCT to measure the absolute velocity of blood at that location expressed in distance over time; determining a calibration factor to convert the blood velocity measured with Doppler flowmetry expressed in Hz to velocity expressed in mm/s; and using the calibration factor to convert the blood velocity measured with Doppler flometry to blood velocity expressed in distance over time.

In one example, a method for determining a calibration factor in Doppler flowmetry velocity measurements in the living eye includes imaging the eye with Doppler flowmetry and processing the data to obtain blood velocity, volume, and flow maps using Doppler flowmetry formulas that provide velocity as a mean frequency expressed in Hz, and volume and flow in arbitrary units. A selected blood vessel is probed with Doppler OCT to measure the absolute velocity of blood at that location expressed in distance over time by scanning the OCT beam in a circular pattern, intersecting the blood vessel twice, identified by two spots in the OCT image, measuring the height difference of the two spots, calculating the angle between the blood vessel and the OCT beam using the measured height difference, and calculating the absolute blood velocity using the angle between the blood vessel and the OCT beam and the axial velocity component obtained from Doppler OCT. A resulting calibration factor can be used to convert the velocity measured with Doppler flowmetry expressed in Hz to velocity expressed in distance over time.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 shows an ultra-widefield velocity montage of the left eye of a normal volunteer illustrating visualization of vortex veins without injecting contrast agent;

FIG. 2 illustrates a LSDF velocity map with potential locations of the OCT probing beam;

FIG. 3 is a diagram of the scanning geometry;

FIG. 4 shows examples of OCT images of a glass tube at different tilt angles embedded in scattering medium;

FIGS. 5A-B show examples of LSDF images (SLO-left and Velocity-right) illustrating the flow in the glass tube and the location of the OCT circular scan;

FIGS. 6A-B show the parabolic fit of the un-wrapped phase images;

FIG. 7 shows the results of tube diameter measurement with OCT;

FIG. 8 shows the results of angle measurement with OCT;

FIG. 9 shows the results of velocity measurement with OCT;

FIG. 10 shows the results of the volumetric flow rate measurement with OCT;

FIG. 11 shows the results of Velocity measured with LSDF;

FIGS. 12A-B illustrate various LSDF power spectra across the flow tube;

FIG. 13 shows changes of the power spectrum shape with increasing flow velocity;

FIG. 14 shows a fit of the LSDF measured Velocity with an error function;

FIG. 15 shows the calibrated LSDF Velocity;

FIGS. 16A-B illustrate a fit of LSDF measured Volume (A) and the tube diameter measurements (B); FIG. 17 shows the LSDF measured flow rate as a function of the set flow rate;

FIG. 18 shows the LSDF measured flow rate as a function of the OCT measured flow rate;

FIG. 19 shows examples of OCT reflectivity and phase images and the corresponding scan diagram in the small circular scan configuration;

FIG. 20 illustrates the LSDF Velocity image with the location of the OCT circular scan and the corresponding OCT images with the two spots automatically identified by the processing software;

FIG. 21 is a block diagram depicting in one example, the primary components associated with a combined LSDF/OCT system;

FIG. 22 is a diagram of the optical components of the system of FIG. 21; and

FIGS. 23A and B are schematic views of an exemplary LSDF/OCT instrument.

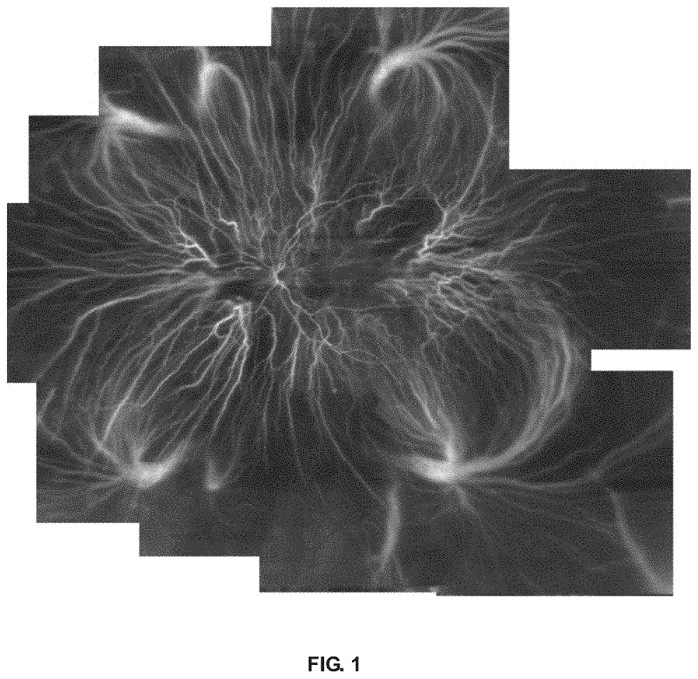

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

In retinal blood flow Doppler (LSDF) imaging, the measurements are given in arbitrary units (Volume and Flow) or Hz (Velocity) and their conversion factor to the correct parameters claimed to be measured were rather elusive so far. The measurements seem to depend on a lot of uncontrolled factors, are operator dependent among other things, and cannot reliably support longitudinal studies on the same eye or comparisons among individuals.

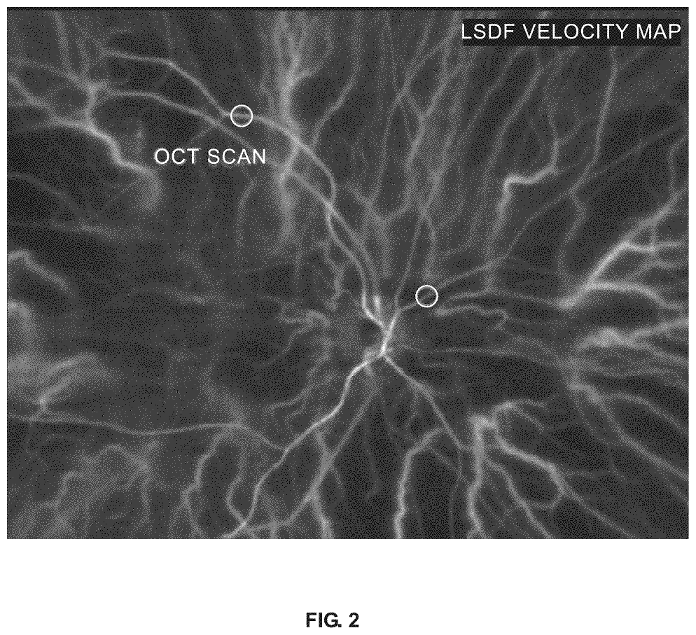

Disclosed here is a method to calibrate the LSDF velocity map by determining the calibration factor from Hz to mm/s based on a local OCT measurement as illustrated in FIG. 2. Potential OCT probe beam circular scan locations are shown as small white circles.

Before performing measurements on human volunteers, we first tested the ability of the OCT system to quantify the flow velocity and the geometric dimensions of the flow channel, as described below. We used a cylindrical glass tube embedded in a scattering medium for a realistic testing of the quantitative flow measurement in a configuration that resembles the flow in a human eye. The flow channel was mounted on a rotation stage that allowed for controlled orientation of the flow with respect to the laser beam and on a micrometric translation stage for placing it in the focal plane of a 25 mm focal length lens. This arrangement acts as a model eye with the lens simulating the eye lens. Measurements were also made holding the flow channel stationary and changing the offset voltage of the scan to change the orientation of the scan vertically as one would do in a real eye to reposition the scan on a blood vessel. The liquid flown through the microfluidic channel was milk diluted in water.

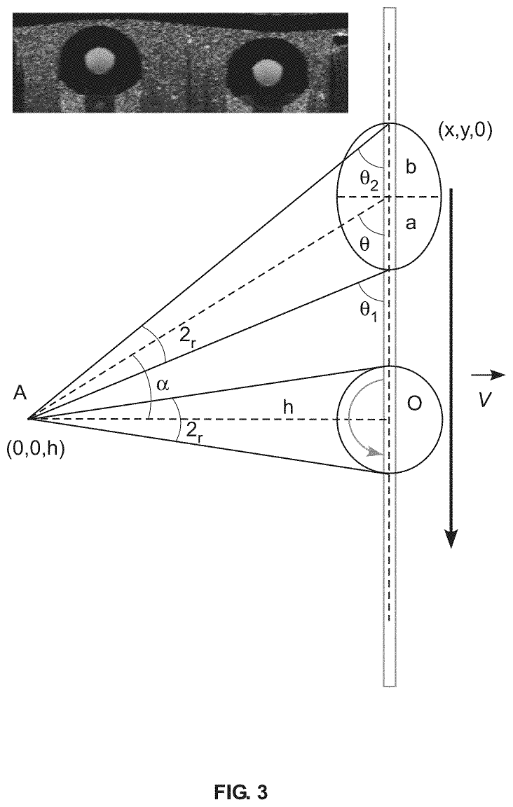

The inside diameter of the cylindrical glass tube was 198 μm. The tube was connected to a syringe pump using silicone tubing to which was glued with epoxy. For a very stable phantom that could be used repeatedly over longer periods of time we used 3 μm aluminum oxide powder mixed in transparent silicone sealant. The scanning geometry is shown in FIG. 3. The OCT beam was scanned over the surface of a cone with the pivot point A. The scan intersects the flow twice and the height difference of the two spots is used to calculate the angle between the flow and the laser beam.

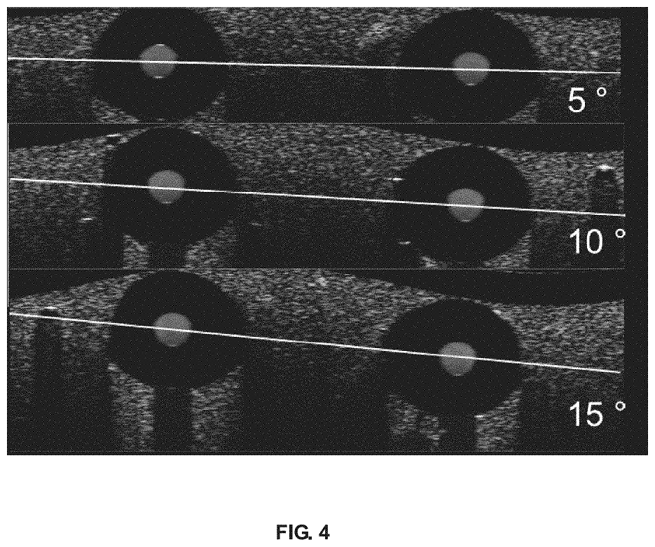

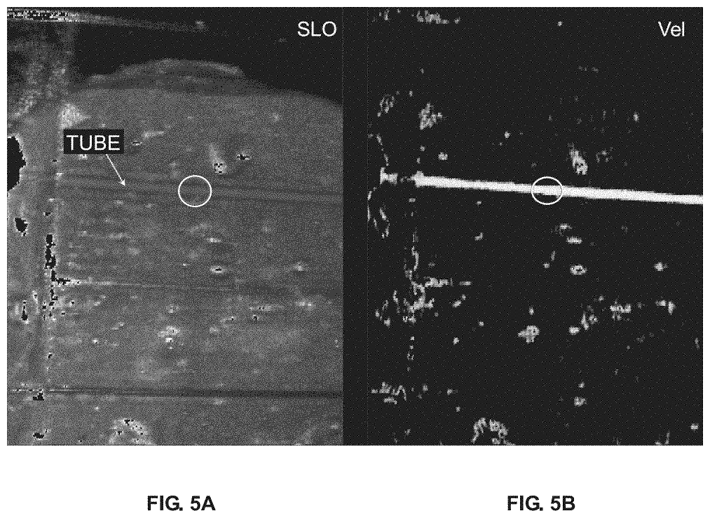

FIG. 4 shows examples of the OCT images from the same tube with different tilt angles. LSDF measurements were also performed and an example is shown in FIG. 5 where the white circle illustrates the position of the OCT circular scan with respect to the tube.

The dark ring around the spots in FIG. 4 is the glass wall of the tube which is clear without any scattering particles. Doppler OCT calculation within the two white spots provides the Doppler phase shift AO (in radians) due to the flow of the diluted milk through the tube. This phase shift provides the axial component of the flow velocity, along the OCT beam. The vertical position of the two spots is used to calculate the tilt angle θ between the flow (tube) and the OCT beam which is then used to calculate the absolute velocity of the flow in each pixel using the following formula:

$$V = \frac{λ}{4 π n Δ T} \frac{Δ∅}{cos θ} \qquad ( 1 )$$

where: λ=1.06 μm is the OCT central wavelength, n=1.33 is the refractive index of water, and ΔT= 1/70 ms is the time between consecutive A-lines (for 70 kHz A-line rate).

200 circular OCT scans were acquired and the phase calculations were averaged over these 200 scans assuming constant velocity flow during the data acquisition time (˜2.86 s). The total angle of the cone scanning geometry was approximately 2° (1° half angle).

One issue with Doppler OCT is that the phase calculation involves an arctangent which wraps at ±π/2. As the velocity and the angle increase, the phase approaches π/2 and then it jumps down to −π/2 resulting in alternating positive and negative rings. A useful procedure that provides rapid automatic un-wrapping of 2D phase images is based on least-squares, iteration and calibration to phase derivatives is described in Xia, H., et al., Phase calibration unwrapping algorithm for phase data corrupted by strong decorrelation speckle noise, Opt. Express 2016. 24(25): p. 28713-28730 and Xia, H., et al., Non-invasive Mechanical Measurement for Transparent Objects by Digital Holographic Interferometry Based on Iterative Least-Squares Phase Unwrapping, Experimental Mechanics, 2012. 52(4): p. 439-445 incorporated herein by this reference.

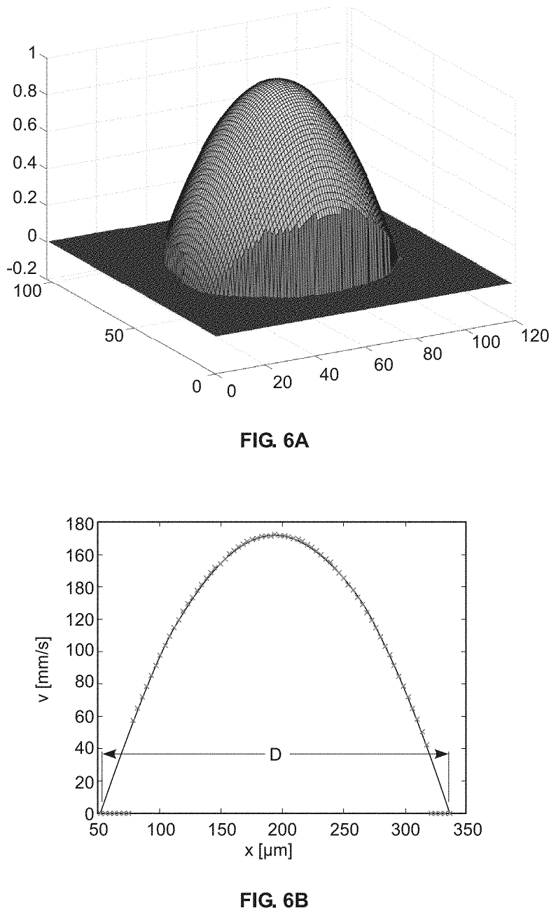

The OCT reflectivity images shown in FIG. 4 allow for segmentation of the tube position and calculation of the vertical location of each of the two spots. The parabolic fit of the Doppler phase shift shown in FIG. 6 also enables localization of the center of the flow. Either one can be used for angle calculation.

FIG. 6B shows the parabolic fit in the vertical (axial) direction which enables calculation of the tube diameter D from the two positions where the parabola gets to zero as there is no flow at the tube wall. A few pixels on both sides of the parabola were removed because they are affected by intensity and phase artifacts due to reflections at the liquid/glass interface.

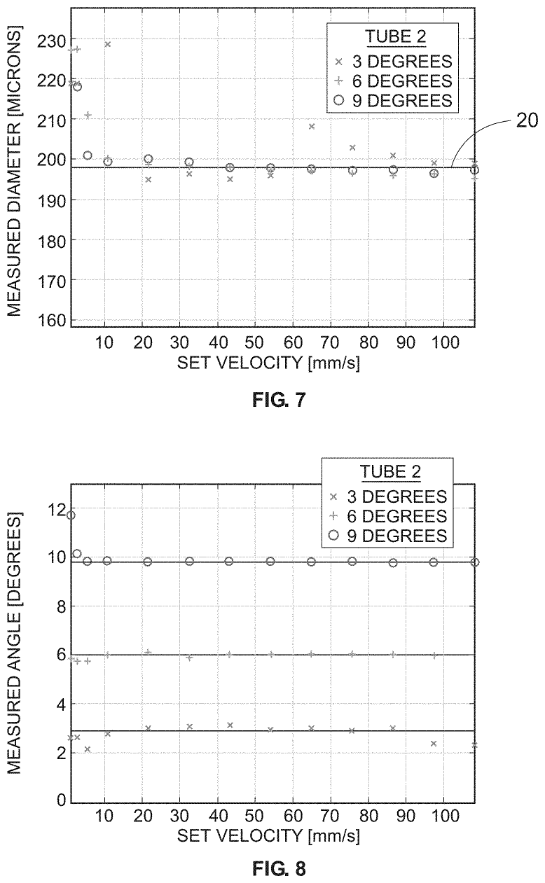

FIG. 7 shows the results of tube diameter measurement with OCT and the three orientations over the entire range of set velocities up to 130 mm/s. The x symbol was used for 3° tilt, the + sign for 6° and the o symbol for 9° tilt. The line 20 is set for the actual value of the tube diameter as provided by the manufacturer with an uncertainty of ±5 μm. It should be noted that most of the measurements are within ±5% of the expected value with the exception of low velocity values below 10 mm/s where the errors are larger. The results are slightly less precise for 3° which is expected since the Doppler shift calculation is most affected by phase noise when the beam is close to normal to the flow.

The measurement of the absolute velocity is based on Eq. 1 which requires estimation of the angle θ between the flow and the laser beam. The results for the angle measurement are shown in FIG. 8. As mentioned above, the flow channel was mounted on a rotation stage that allowed for controlled orientation of the flow with respect to the laser beam. The intended angle between the axis of the scanning cone and the tube was set at 3°, 6°, and 9°, as it is called out in all the plots (3°-x, 6°-+, 9°-o). However, the set rotation was with respect to the vertical axis and the tube was not always orthogonal to this axis. There might have been slight misalignments between the tube and the slide holding the tube and between the slide and the rotation stage and/or the axis of the scanning cone might not have been always perfectly horizontal. Therefore, some additional compound angles which were not easy to measure slightly affected the actual angle between the tube and the beam.

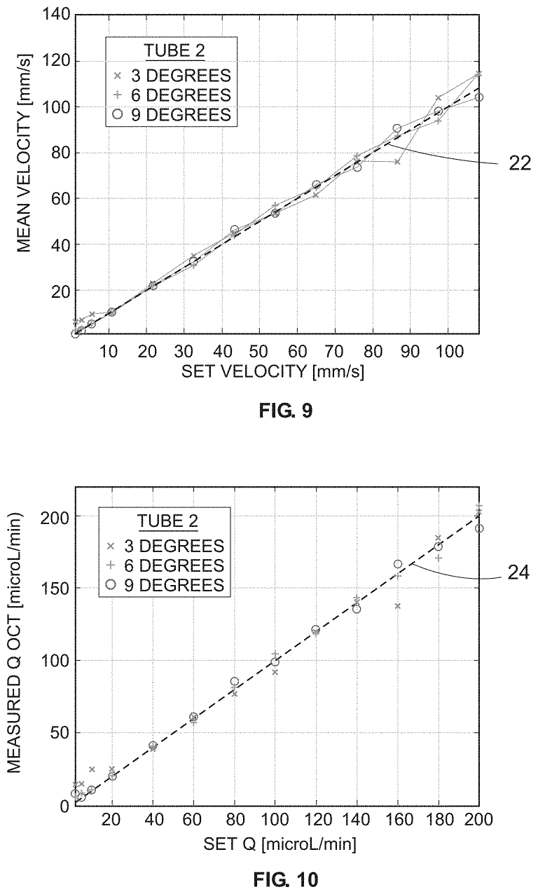

The line for each measurement set in FIG. 8 is a fit through the data and therefore an estimate of the actual angle between the flow tube and the OCT beam. Most of the times it is close to the set value, but not exact. However, the measured value is remarkably constant across the entire set velocity range (with some exceptions below 5 mm/s). This angle value was used for velocity calculation shown in FIG. 9.

The results of the velocity measurement from OCT data are shown in FIG. 9 with the same colors and symbols as mentioned above. There is also a line 22 in that plot, generally buried under data, as a guide for one-to-one correspondence between the measured (vertical) and set (horizontal) values. The experimental values are remarkably close to this guide with small errors noted again for 3° as expected.

The volumetric flow rate Q can now be calculated as:

$$Q = \frac{π {D}^{2}}{4} V \qquad ( 2 )$$

using the measured diameter D and the measured velocity V as shown above. The results for the three orientations are shown in FIG. 10. The line 24 is again a one-to-one correspondence between the measured and the set values as a guide. These results provide validation of the ability of the Doppler OCT technique used here to precisely quantify flow parameters in a configuration relatively similar to the blood vessels embedded in retinal tissue.

Line-scanning Doppler flowmetry measurements were performed for all orientations and set velocities as in the OCT measurements simultaneously with the OCT scans. Ten datasets were acquired for each measurement configuration and were averaged assuming constant flow during the measurement time (˜6.26 s). The measurements were also averaged along the tube over a set distance which was selected to avoid strong specular reflections which generally skew the results.

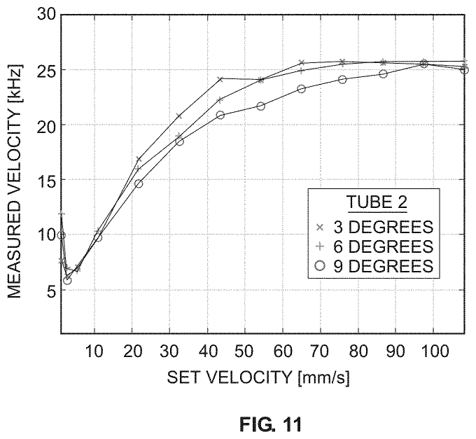

FIG. 11 shows the results of the Velocity measurements with LSDF for the three orientation angles. One can notice in FIG. 11 that measured Velocity shows a linear increase for a certain set velocity range, and then it saturates, it flattens out. For visualization purposes it is fine, but for quantification purposes it is a problem and it needs to be addressed. Below we present a potential explanation of the phenomenon observed and a solution to velocity quantification using OCT.

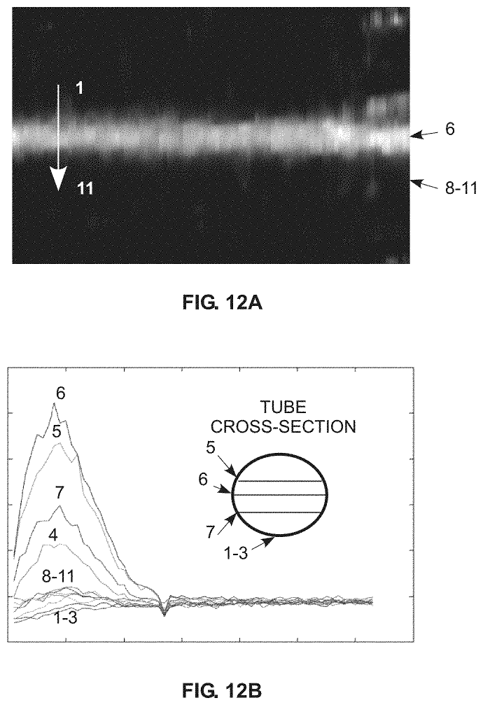

To better understand the issue, we looked at the changes of the power spectrum with increasing flow velocity. FIG. 12A shows the realigned (flattened) volume image of the tube. FIG. 12B shows a few power spectra across the flow. The spectra were averaged along the flow and were normalized to the average spectrum over the entire image. The average spectra 5 and 6 are close to the center of the tube, 4 and 7 are above or below the center, while 1-3 and 8-11 are at the edge or outside the tube. There are 128 sampling points in the time trace of intensity fluctuations and the Fourier transform of the time trace provides 64 points in the power spectrum (the horizontal axis in FIG. 12) as frequencies. The maximum measurable frequency is 45 kHz (half the sampling rate) shown at point 64. The minimum measurable frequency is 0.7 kHz (point 2, point 1 being the DC).

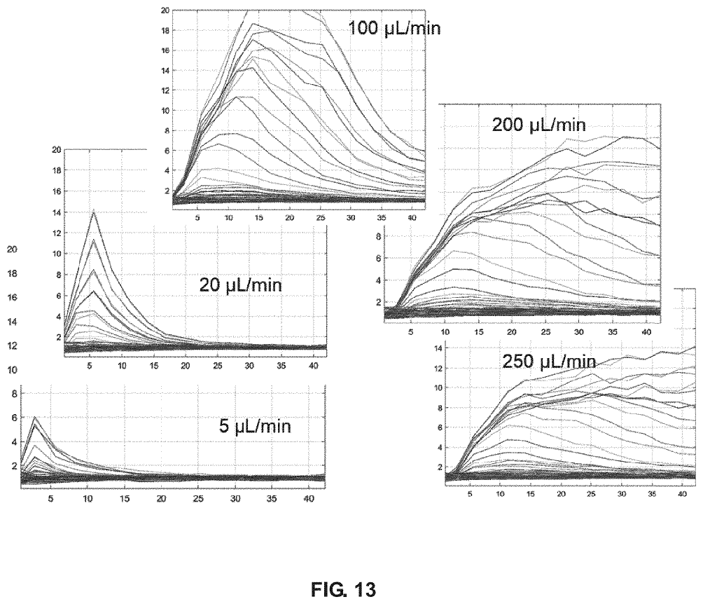

The velocity is defined in LDF as the mean frequency within the measurement range which works well for slow flow. The problem starts as the flow speed increases and the bell shape starts to move to the right. At some point it starts clipping on the right side as one can see in the sequence shown in FIG. 13 as the set velocity increases. For slow flow, the largest Doppler shift frequencies are still within the measurement window and the mean frequency can be properly evaluate from the power spectrum. This corresponds to the linear increase in FIG. 11 for slow flow. As the flow speed increases, the Doppler shift frequencies increase and the bell shape of the power spectrum moves to the right. At some point, the largest frequency shifts exceed the maximum measurable frequency with the current system of 45 kHz. As the power spectrum is clipped on the right side and a significant part of the power spectrum cannot be measured, the average frequency of the measured spectrum is clearly underestimated and the dependency shown in FIG. 11 flattens out at a value of the order of 25 kHz.

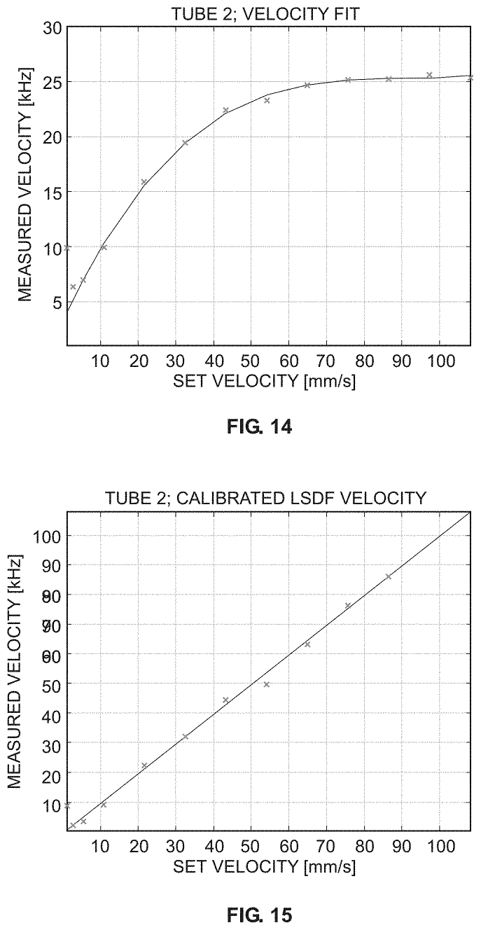

With this explanation in mind, we developed a solution for proper quantification of Velocity measurement with LSDF. The average of the three orientations is shown in FIG. 14 together with a fit with an error function that seems most appropriate for the data. Since the velocity measurement was validated above with OCT measurements against the set velocity, this fit of LSDF Velocity data as a function of the set velocity can now be used to “correct”, to calibrate the LSDF Velocity measured in kHz into mm/s values. Therefore, there is no single linear calibration factor from Hz to mm/s. We can determine the inverse function of this fit and then extract the correct velocity value [mm/s] for any new measured LSDF Velocity [Hz] value as a lookup table procedure.

It should be noted here that in the plateau region, which is not perfectly flat, there is still a slow trend up, small errors in the Velocity measurement result in large swings in the calibrated velocity value. It should also be obvious that measured Velocity values larger than the largest value of the fitting curve generate invalid correction and cannot be used.

Using the inverse function of the fit shown in FIG. 14 we can convert the measured Velocity [kHz] values into calibrated velocity [mm/s] value. The results are shown in FIG. 15.

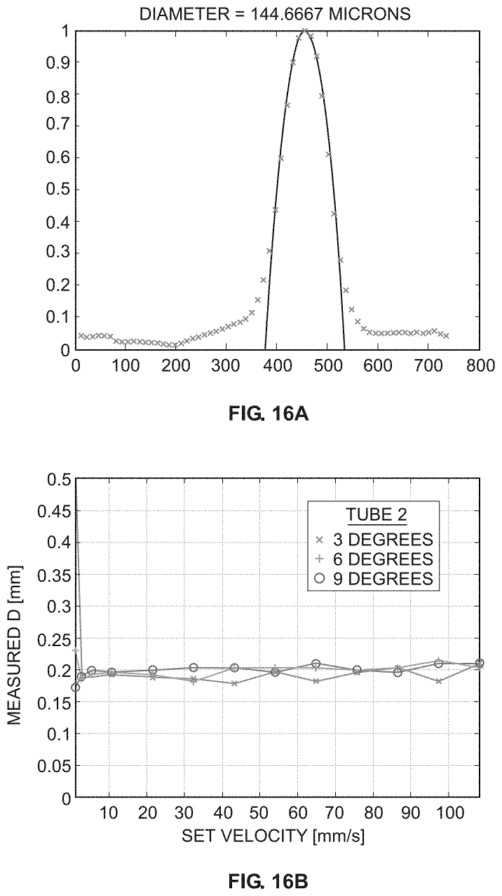

Similarly to the estimation of the tube diameter from OCT data, we can evaluate the diameter from LSDF data. The need for that stems from the fact that the OCT measurement for flow analysis is a local measurement, over the small circular scan, while LSDF is providing a large area map of the flow. Lateral average of the Volume image provides a profile of the Volume across the tube as shown in FIG. 16A. Volume is calculated as the total power of intensity fluctuations relative to the DC (non-Doppler shifted) value and is generally regarded as the number of scatterers within the laser interrogation volume. Therefore, Volume is expected to drop to zero at the tube wall. The profile in FIG. 16A is fit with a parabolic profile and the tube diameter is estimated from the distance between the two points on the horizontal axis (scaled in μm) where the fit drops to zero.

The results of the tube diameter measurement with LSDF for the three orientations are shown in FIG. 16B. As with other measurements, the errors are more significant below 5 mm/s set velocity.

Having both the velocity (calibrated) and the diameter (average over the three orientations), we can calculate the volumetric flow rate Q using equation 2 and we can compare it with the set flow rate and the OCT measured flow rate. FIG. 17 shows the LSDF flow rate as a function of the set flow rate. The results show a quite remarkable linear dependence.

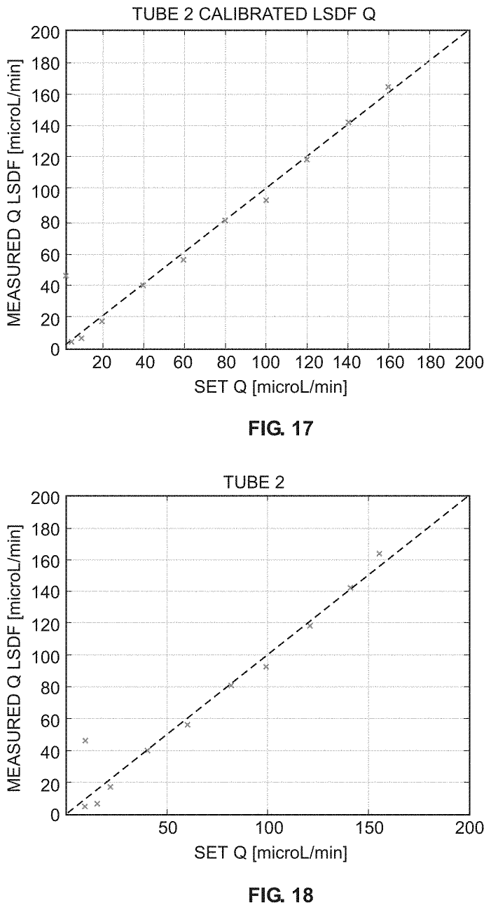

FIG. 18 shows the comparison between the volumetric flow rate measured with OCT and LSDF and it clearly validates the concept of calibrating the LSDF measurements using the OCT measurements.

The ability of the Doppler OCT technique used here to precisely quantify flow parameters in a configuration similar to the blood vessels embedded in retinal tissue has been tested and validated using a glass tube at three different orientations with respect to the OCT beam. OCT is used as a local calibration probe. LSDF is used to generate large area maps of the retinal blood low. LSDF can also be used to measure the diameter and the velocity of the flow and the OCT measurement can be used to calibrate the LSDF measured velocity into the proper unit of measurement [mm/s].

Following the experiments described above on microfluidics that validated the ability of the combined OCT-LSDF technology to quantify the flow parameters, preliminary demonstration on the eye of human volunteers and on rats with retinal degeneration was performed.

Ten LSDF large area raster scans were acquired simultaneously with 200 circular OCT scans. The OCT scans were positioned to intersect a retinal blood vessel identified live in the SLO image or in the LSDF Velocity map following the concept shown in FIG. 2. Since the optimum position of the OCT circular scan is such that it intersects the blood vessel along a diameter of the circle, the distance between the two spots should be approximately half the lateral image size. Therefore, the operator adjusts the relative position of the circular scan with respect to the blood vessel watching the position of these two spots after an initial alignment brings the circle close to the blood vessel in the SLO/Velocity image.

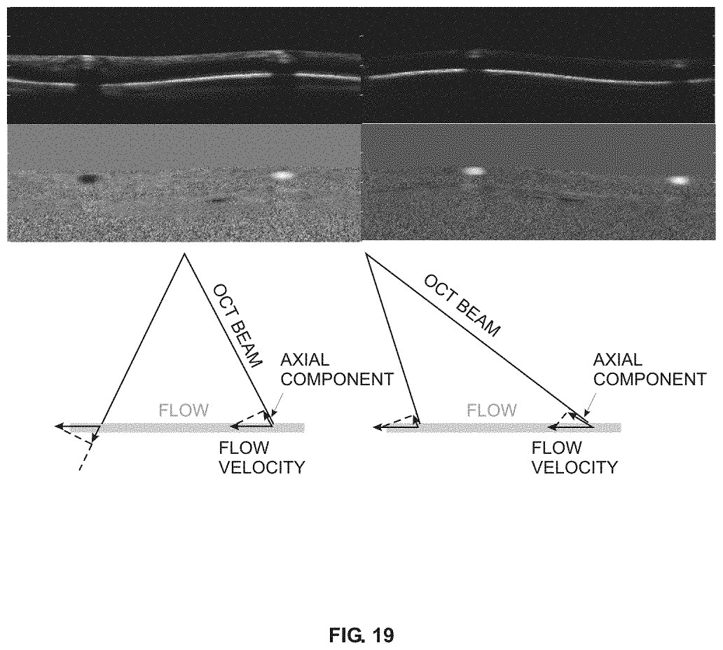

FIG. 19 shows two examples of OCT reflectivity (top)/phase (center) images that were aligned and averaged over the 200 acquired scans. One can clearly identify the two spots corresponding to the location of the blood vessel both in the reflectivity and in the phase image.

It should be noted here that the left image shows a white and black spot while the right image shows two white spots. White vs. black indicates that the axial component of the flow velocity is directed up or down. The bottom row in FIG. 19 shows an illustration of the scan geometry that explains this effect. The angles in these diagrams are greatly exaggerated for illustration purposes. In a real scan configuration, the total angle at the top of the scanning cone is ˜2°. The left image in FIG. 19 illustrates the situation when the axis of the scanning cone is almost normal to the flow and therefore the axial velocity component (along the OCT beam) can be towards or away from the tip of the cone and can have a positive or a negative Doppler shift (phase shift). The right image in FIG. 19 shows a situation in which both velocity components are towards the tip of the cone having the same sign. One can notice that the black/white spots in the left image have approximately the same height (distance from the scan pivot point) while the two white spots in the right image have different heights, both situation resembling the geometry shown in the diagrams in FIG. 19.

The example shown in FIG. 19 demonstrates the advantage of the small circular scan that intersects the blood vessel twice at different angles as compared to a single B-scan that intersects the blood vessel only ones. A single B-scan normal to the blood vessel cannot measure Doppler shift. The circular scan used here can never have both intersections normal to the vessel simultaneously; therefore, the circular scan configuration can provide the absolute velocity in any orientation of the flow velocity.

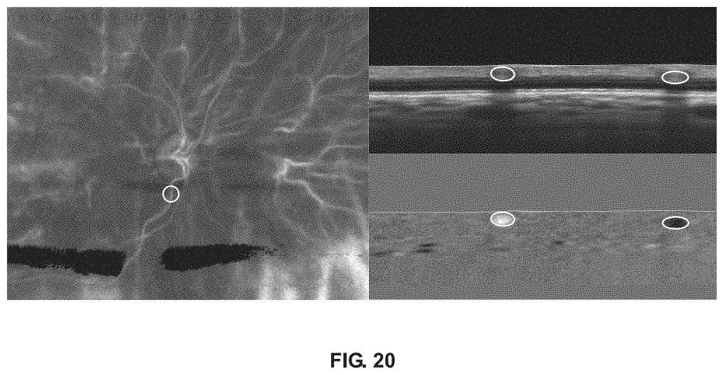

Processing software automatically identifies the two spots (illustrated in FIG. 20), calculates the height difference and therefore the tilt angle as described above for the glass tube. A parabolic fit is performed on the identified phase spots to measure the diameter of the blood vessel and the axial velocity component. Knowing the tilt angle and the axial velocity component, the absolute velocity is calculated. The circular overlay in the LSDF Velocity image (left in FIG. 20) allows us to estimate the LSDF Velocity at the same location as that of the OCT scan and therefore to calibrate the LSDF Velocity image using the calibration function determined from the microfluidics calibration procedure.

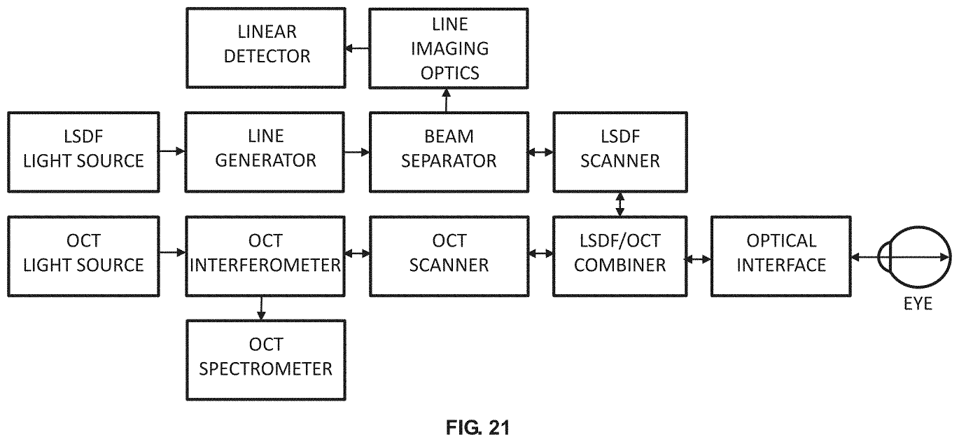

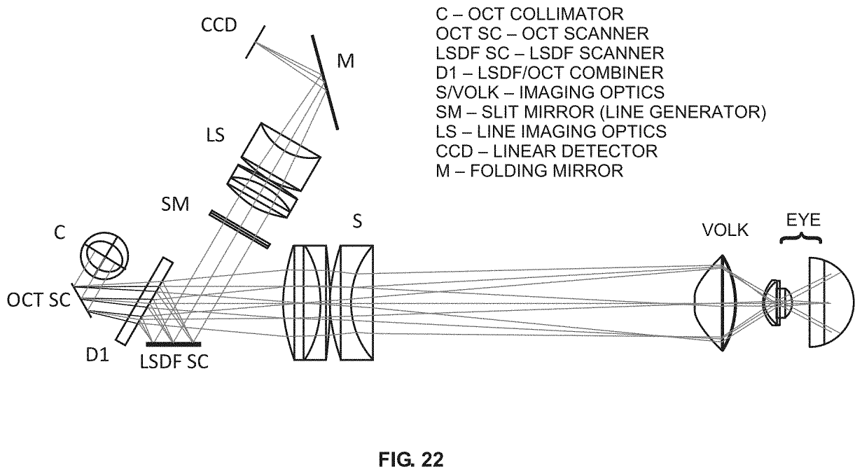

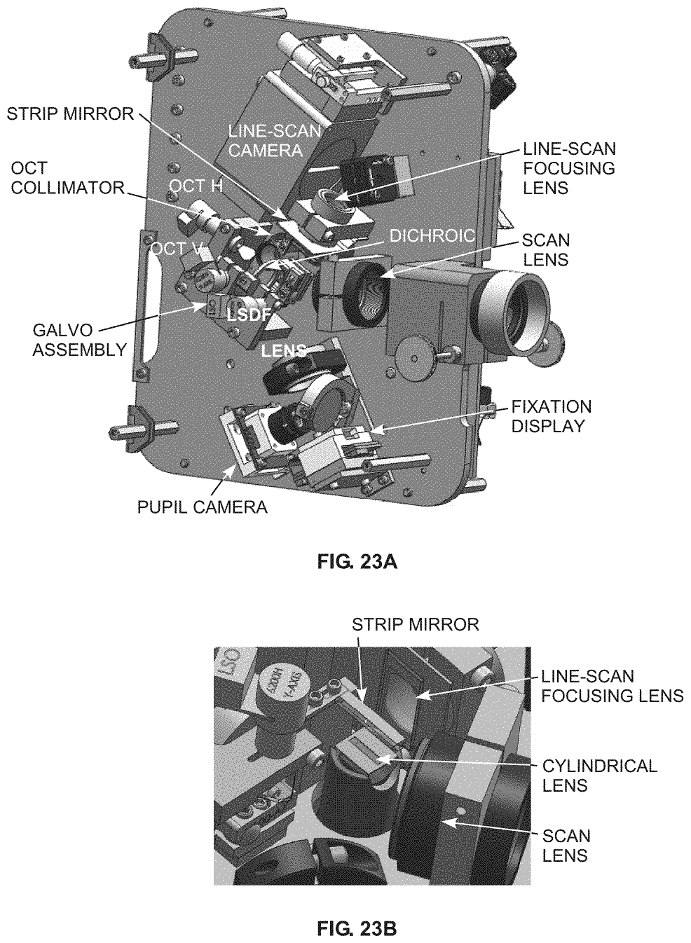

In one example, the imaging instrument is illustrated through a block diagram in FIG. 21, an optical design diagram in FIG. 22 and CAD diagrams in FIG. 23. The LSDF/OCT imaging system is based on two main optical paths: LSDF and OCT imaging paths. The LSDF imager uses a fixed cylindrical lens and a slit/strip mirror to generate a line of light which is scanned with a single galvanometer scanner (LSDF scanner). The OCT imager uses a pair of galvanometer scanners to steer the OCT beam independently from LSDF. The LSDF and OCT optical paths are then combined at a dichroic beam splitter D1. The common path proceeds to the scan lens group S and to an ophthalmoscopic lens (VOLK lens up to 66 D) which together with the eye optics relays the image plane to the retina.

The LSDF path begins with the LSDF collimator (located on the back of the plate shown in FIG. 23) which produces a 10 mm beam. The LSDF beam is then passed with a turning mirror to the central section optical plate where a rotary mount holds a 15 mm focal length cylindrical lens which focuses the beam to a line near the aperture-separating strip mirror SM as shown in FIG. 23B. This line is scanned by the LSDF galvo, reflected by the LSDF/OCT combining dichroic D1, and relayed by the scanning/imaging optics (and the eye optics) to the retina.

The strip mirror SM is a 2 mm width section of a 1 in. plane mirror that reflects the focused LSDF line beam and passes the returning reflection from retinal focal plane over the whole aperture. The LSDF scanner is conjugate to the ocular pupil (approximately 3 to 5 mm from the corneal surface) while the strip mirror is nearly conjugate to the corneal surface. This feature ensures that reflections from cornea are efficiently stopped by the strip mirror leaving as much as 80% of the collection aperture for gathering the reflected light from the retina, and therefore usually requiring no other means of eliminating unwanted reflections (e.g., polarizers).

The LSDF optical detection path begins from the eye model at right and proceeds through the VOLK ophthalmic lens; through the front scan lens group S (achromat and negative meniscus); reflects from the LSDF/OCT beam-combining dichroic D1 and the LSDF scanner; passes the strip mirror SM to the line-scan focusing lens LS; and reflects from the turning mirror M to the line-scan camera (CCD).

The OCT imaging path consists of a triplet collimator C (Thorlabs—25 mm focal length and ˜5 mm beam diameter) and a pair of x-y galvo scanners SC (OCT H and V). The collimated beam passes through the LSDF/OCT beam-combining dichroic D1 and then to the retina through the imaging optics common to the LSDF path. The OCT collimator C and the compound lens S define the focal plane to which the imaging path of the LSDF channel needs to be focused during instrument alignment to ensure that the depth range of the LSDF and the OCT channels overlap in the retina. The OCT detection path also includes the OCT interferometer and the spectrometer.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A method for determining a calibration factor in Doppler flowmetry velocity measurements in the living eye, the method comprising:
imaging the eye with Doppler flowmetry;
processing the data to obtain blood velocity, volume, and flow maps using Doppler flowmetry formulas that provide velocity as a mean frequency expressed in Hz, and volume and flow in arbitrary units;
probing a selected blood vessel with Doppler OCT to measure the absolute velocity of blood at that location expressed in mm/s; and
determining a calibration factor to convert the velocity measured with Doppler flowmetry expressed in Hz to velocity expressed in mm/s.

2. The method in claim 1 wherein the living eye is a human eye or an animal eye.

3. The method in claim 1 wherein the Doppler flowmetry method is line-scanning Doppler flowmetry (LSDF).

4. The method in claim 1 wherein the calibration factor is calculated using LSDF and OCT measurements at the same location in the retina and at the same time.

5. The method of claim 1 in which probing a selected blood vessel with Doppler OCT includes:
scanning the OCT beam in a circular pattern;
intersecting the blood vessel twice, identified by two spots in the OCT image;
measuring the height difference of the two spots;
calculating the angle between the blood vessel and the OCT beam using the measured height difference; and
calculating the absolute blood velocity using the angle between the blood vessel and the OCT beam and the axial velocity component obtained from Doppler OCT.

6. The method in claim 5 wherein the circular OCT scan is generated by scanning an OCT beam on the surface of a cone with the apex in the center of the eye pupil.

7. The method in claim 6 wherein the cone angle is approximately 2 degrees.

8. The method of claim 1 in which obtaining blood volume includes:
fitting a profile of the Doppler flowmetry data in a plane perpendicular to the blood vessels with a parabolic function; and
calculating the diameter of the blood vessel as the distance between the two points where the parabolic fit function is zero.

9. The method in claim 8 wherein the Doppler flowmetry data is a velocity map.

10. The method in claim 8 wherein the Doppler flowmetry data is a volume map.

11. The method in claim 8 wherein the Doppler flowmetry data is a flow map.

12. The method of claim 8 in which calculating the cross-sectional area of the blood vessels includes using a calculated blood vessel diameter.

13. The method of claim 12 in which obtaining the volumetric flow map expressed in mm³/s, comprises multiplying the calibrated velocity map by the calculated cross-sectional area map.

14. A method of determining blood velocity measurements in the living eye, the method comprising:
imaging the eye with Doppler flowmetry;
obtaining blood velocity, volume, and flow maps using Doppler flowmetry formulas that provide velocity as a mean frequency expressed in Hz, and volume and flow in arbitrary units;
probing a selected blood vessel with Doppler OCT to measure the absolute velocity of blood at that location expressed in distance over time;
determining a calibration factor to convert the blood velocity measured with Doppler flowmetry expressed in Hz to blood velocity expressed in distance over time; and
calculating the blood velocity in distance over time using the calibration factor.

15. A method for determining blood velocity measurements in the living eye, the method comprising:
imaging the eye with Doppler flowmetry;
obtaining velocity, volume, and flow maps using Doppler flowmetry formulas that provide blood velocity as a mean frequency expressed in Hz and volume and flow in arbitrary units;
probing a selected blood vessel with Doppler OCT to measure the absolute velocity of blood at that location expressed in distance over time;
determining a calibration factor to convert the blood velocity measured with Doppler flowmetry expressed in Hz to velocity expressed in mm/s; and
using the calibration factor to convert the blood velocity measured with Doppler flometry to blood velocity expressed in distance over time.

16. The method in claim 15 wherein the living eye is a human eye or an animal eye.

17. The method in claim 15 wherein the Doppler flowmetry method is line-scanning Doppler flowmetry (LSDF).

18. The method in claim 15 wherein the calibration factor is calculated using LSDF and OCT measurements at the same location in the retina and at the same time.

19. The method of claim 15 in which probing a selected blood vessel with Doppler OCT includes:
scanning the OCT beam in a circular pattern;
intersecting the blood vessel twice, identified by two spots in the OCT image;
measuring the height difference of the two spots;
calculating the angle between the blood vessel and the OCT beam using the measured height difference; and
calculating the absolute blood velocity using the angle between the blood vessel and the OCT beam and the axial velocity component obtained from Doppler OCT.

20. The method in claim 18 wherein the circular OCT scan is generated by scanning an OCT beam on the surface of a cone with the apex in the center of the eye pupil.

21. The method in claim 19 wherein the cone angle is approximately 2 degrees.

22. The method of claim 15 in which obtaining blood volume includes:
fitting a profile of the Doppler flowmetry data in a plane perpendicular to the blood vessels with a parabolic function; and
calculating the diameter of the blood vessel as the distance between the two points where the parabolic fit function is zero.

23. The method in claim 2 2 wherein the Doppler flowmetry data is a velocity map.

24. The method in claim 22 wherein the Doppler flowmetry data is a volume map.

25. The method in claim 22 wherein the Doppler flowmetry data is a flow map.

26. The method of claim 22 in which calculating the cross-sectional area of the blood vessels includes using a calculated blood vessel diameter.

27. The method of claim 25 in which obtaining the volumetric flow map comprises multiplying the calibrated velocity map by the calculated cross-sectional area map.

28. A method for determining a calibration factor in Doppler flowmetry velocity measurements in the living eye, the method comprising:
imaging the eye with Doppler flowmetry;
processing the data to obtain blood velocity, volume, and flow maps using Doppler flowmetry formulas that provide velocity as a mean frequency expressed in Hz, and volume and flow in arbitrary units;
probing a selected blood vessel with Doppler OCT to measure the absolute velocity of blood at that location expressed in distance over time by:
scanning the OCT beam in a circular pattern,
intersecting the blood vessel twice, identified by two spots in the OCT image,
measuring the height difference of the two spots,
calculating the angle between the blood vessel and the OCT beam using the measured height difference, and
calculating the absolute blood velocity using the angle between the blood vessel and the OCT beam and the axial velocity component obtained from Doppler OCT; and
determining a calibration factor to convert the velocity measured with Doppler flowmetry expressed in Hz to velocity expressed in distance over time.

29. The method in claim 28 wherein the living eye is a human eye or an animal eye.

30. The method in claim 28 wherein the Doppler flowmetry method is line-scanning Doppler flowmetry (LSDF).

31. The method in claim 28 wherein the calibration factor is calculated using LSDF and OCT measurements at the same location in the retina and at the same time.

32. The method in claim 28 wherein the circular OCT scan is generated by scanning an OCT beam on the surface of a cone with the apex in the center of the eye pupil.

33. The method in claim 32 wherein the cone angle is approximately 2 degrees.

34. The method of claim 28 in which obtaining blood volume includes:
fitting a profile of the Doppler flowmetry data in a plane perpendicular to the blood vessels with a parabolic function; and
calculating the diameter of the blood vessel as the distance between the two points where the parabolic fit function is zero.

35. The method in claim 34 wherein the Doppler flowmetry data is a velocity map.

36. The method in claim 34 wherein the Doppler flowmetry data is a volume map.

37. The method in claim 34 wherein the Doppler flowmetry data is a flow map.
